SEQUENCE LISTING (17 sequences)

SEQ ID NO: 1 - protein, 2 aa, Artificial Sequence
  synthetic
VK

SEQ ID NO: 2 - protein, 5 aa, Artificial Sequence
  synthetic
HYTAV

SEQ ID NO: 3 - protein, 12 aa, Artificial Sequence
  synthetic
HYTAVVKKSSAV

SEQ ID NO: 4 - protein, 7 aa, Artificial Sequence
  synthetic
HYTAVVK

SEQ ID NO: 5 - protein, 12 aa, Artificial Sequence
  synthetic
LDIELVNCDITA

SEQ ID NO: 6 - protein, 7 aa, Artificial Sequence
  synthetic
ELVNCDI

SEQ ID NO: 7 - protein, 12 aa, Artificial Sequence
  synthetic
KLAFTGPIVNGH

SEQ ID NO: 8 - protein, 12 aa, Artificial Sequence
  synthetic
FTGPIVNGHSDE

SEQ ID NO: 9 - protein, 12 aa, Artificial Sequence
  synthetic
TLKDGENVLHYT

SEQ ID NO: 10 - protein, 7 aa, Artificial Sequence
  synthetic
DGENVLH

SEQ ID NO: 11 - protein, 7 aa, Artificial Sequence
  synthetic
NVLHYTA

SEQ ID NO: 12 - protein, 7 aa, Artificial Sequence
  synthetic
NGHSDEL

SEQ ID NO: 13 - protein, 10 aa, Artificial Sequence
  synthetic
KSMCMKLSFS

SEQ ID NO: 14 - protein, 12 aa, Artificial Sequence
  synthetic
HYTAVVKKSSAV

SEQ ID NO: 15 - protein, 7 aa, Artificial Sequence
  synthetic
HYTAVVK

SEQ ID NO: 16 - protein, 7 aa, Artificial Sequence
  synthetic
HYTASSK

SEQ ID NO: 17 - protein, 7 aa, Artificial Sequence
  synthetic
HYTASSK

(19) United States
(12) Patent Application Publication
(10) Pub. No.: US 2003/0109675 A1
(43) Pub. Date: Jun. 12, 2003

(54) DegP protease: cleavage site identification and proteolysis of a natural target in E. coli

(76) Inventors: C. Hal Jones, Corvallis, OR (US); Paul L. Dexter, Corvallis, OR (US); Amy K. Evans, West Linn, OR (US); Dennis E. Hruby, Albany, OR (US)

Correspondence Address: BURNS, DOANE, SWECKER & MATHIS, L.L.P., P.O. Box 1404, Alexandria, VA, 22313-1404, US

(21) Appl. No.: 10/284,252

(22) Filed: Oct. 31, 2002

Related U.S. Application Data
(60) Provisional application No. 60/330,855, filed on Nov. 1, 2001.

(51) Int. Cl. C07K 1/00; C07K 14/00; C07K 17/00

(52) U.S. Cl. 530/350

(57) ABSTRACT

The DegP protease is essential for clearance of denatured or aggregated proteins from the periplasmic space in bacteria. The present invention relates to novel methods for treatment and/or prophylaxis of diseases caused by pilus-forming bacteria by modulating DegP protease activity. Also disclosed is the identification of DegP cleavage sites on PapA pilin subunit and methods for identifying substances which modulate DegP activity. The present invention further provides for polypeptides identified which are cleavable substrates for DegP. The present invention provides for the identification of a polypeptide which enhances DegP protease activity. The current invention facilitates the development of a novel class of anti-infectives targeting DegP protease.

### CROSS-REFERENCE TO RELATED APPLICATION

This application claims priority to United States provisional Application Serial No. 60/330,855, filed Nov. 1, 2001, which is hereby incorporated by reference and relied upon in its entirety.

### BACKGROUND OF THE INVENTION

Pathogenic Gram-negative bacteria cause a number of pathological conditions such as bacteraemia, bacteria-related diarrhea, meningitis, and urinary tract infection, including pyelonephritis, cystitis, and urethritis.

Urinary tract infections (UTIs)are a major cause of morbidity in females. Despite the overall importance of urinary tract infections, few efforts have been directed toward the development of novel strategies for treatment or preventions of these diseases. Currently, conventional antibiotics (e.g., penicillins, cephalosporins, aminoglycosides, sulfonamides, tetracyclines, nitrofurantoin, and nalidixic acid) are employed to treat these infections. It is expected that emerging antibiotic resistance will become a significant obstacle to treating these infections. In fact, multiple antibiotic resistance in uropathogens has already been detected and is increasing. Estimates of the annual cost of evaluation and treatment of women with UTIs exceed one billion dollars. Further, approximately a quarter of the 4 billion dollar annual cost associated with nosocomial infections is a consequence of UTIs. Among Gram-negative bacterial culprits of UTIs, Escherichia coli predominates.

The ability of Gram negative bacteria (e.g., E. coli, Haemophilus influenzae, Salmonella enteriditis, Salmonella typhimurium, Bordetella pertussis, Yersinia pestis, Yersinia enterocolitica, helicobacter pylori, and Klebsiella pneumoniae) to adhere to a variety of epithelial tissues is an important factor in infectability. As one example, E. coli adheres to epithelial cells despite a unidirectional flushing effect of urine from the kidneys.

The initiation and persistence of many bacterial infections such as those described above is thought to require the presentation of adhesins on the surface of the microbe in accessible configurations which promote binding events that dictate whether extracellular colonization, internalization, or other cellular responses will occur. Adhesins are often components of the long, thin filamentous, heteropolymeric protein appendages known as pili, fimbriae, or fibrillae. The bacterial attachment event is often the result of a stereochemical fit between an adhesin frequently located at the pilus tip and specific receptor architectures on host cells, often comprising carbohydrate structures in membrane-associated glyoconjugates.

Uropathogenic strains of E. coli express P pili that bind to receptors present in uroepithelial cells. Adhesive P pili are virulence determinants associated with pyelonephritic strains of E. coli. At least eleven genes are involved in the biosynthesis and expression of functional P pili; the DNA sequence of the entire pap gene cluster has been determined. P pili are composite heteropolymeric fibers consisting of flexible adhesive fibrillae joined end to end to pilus rods. The pilus rod is composed of repeating PapA protein subunits arranged in a right-handed helical cylinder. Tip fibrillae which extend from the distal ends of each pilus rod were found to be composed mostly of repeating subunits of PapE arranged in an open helical conformation. The PapG adhesin was localized to the distal ends of the top fibrillae, a location which is assumed to maximize its ability to recognize glycolipid receptors on eukaryotic cells. Two minor pilus components, PapF and PapK, are specialized adaptor proteins found in the top fibrillum. PapF links the adhesin moiety to the fibrillum while PapK joins the fibrillum to the pilus rod. The composite architecture of the P pilus fibre reveals the strategy used by uropathogenic E. coli to present the PapG adhesin to eukaryotic receptors. The rigid PapA rod extends the adhesin away from interference caused by LPS and other components at the bacterial cell surface while the flexible fibrillum allows PapG steric fredom to recognize and bind to a digalactoside moiety on the uroepithelium.

The DegP (HtrA, Protease Do) protease is a multifunctional protein essential for the removal of misfolded and aggregated proteins in the periplasm of E. coli. DegP is one of several dozen proteases in E. coli and is known to have homologues in virtually all Gram-negative bacteria, cyanobacteria, mycobacteria, as well as in higher order organisms: yeast and man (Pallen and Wren, Mol. Microbiol. 26(2):209-221 (1997)). DegP homologues have recently been described in a number of Gram-positive bacteria, including Enterococcus faecalis, Lactococcus lactis, Streptococcus pneumoniae, S. gordonii, S. pyogenes, S. mutans, Lactobacillus helveticus, Bacillus subtilis, and Staphylococcus aureus (2 homologues) (Jones et al., Infect. & Immun. 69(9):5538-5545 (2001); Noone et al., J. Bacteriol. 182:1592-1599 (2000); Poquet et al., Mol. Microbiol. 35:1042-1051(2000); Smeds et al., J. Bacteriol. 180:6148-6153 (1998)). There are also two homologues of DegP, DegQ and DegS, in E. coli (Kolmar et al., J. Bacteriol. 178:5925-5929 (1996); Waller and Sauer, J. Bacteriol. 178:1146-1153 (1996)). Kolmar et al. (1996) have suggested that DegQ possesses similar specificity to DegP. DegP has been rediscovered several times as is revealed by the nomenclature (Miller, “Protein Degradation and Proteolytic Modification” in Escherichia coli and Salmonella: Cellular and Molecular Biology, F. C. Niehardt, ed., ASM Press, Washington D.C., pp. 938-954 (1996); Pallen and Wren, 1997; Seol et al., Biochem. Biophys. Res. Comm. 176:730-736 (1991). The DegP (degradation) nomenclature refers to the initial mapping of a mutation in E. coli that allowed accumulation of unstable fusion proteins in the periplasm (Strauch and Beckwith, Proc. Natl. Acad. Sci. USA 85:1576-1580 (1988), Strauch et al., J. Bacteriol. 171:2689-2696 (1989)). The Htr (heat shock regulated) designation indicates that a transposon insertion in the same gene resulted in a temperature sensitive growth phenotype (Lipinska et al., Nucl. Acids Res. 16:10053-10066 (1988)). Lastly, DegP was also designated protease “Do” again as a mutation that conferred a temperature sensitive growth phenotype in E. coli (Seol et al., 1991). The “DegP designation will be used throughout this specification when referring to the E. coli protein.

Purified DegP exhibited functional protease activity in in vitro assays using casein as a substrate, although its activity on this substrate was weak (Lipinska et al., J. Bacteriol. 172:1791-1797 (1990)). Lipinska et al. (Lipinska et al., 1990) demonstrated that the activity on casein was inhibitable by diisopropyl fluorophosphate and not by any other known protease inhibitors, suggesting that DegP contains an active site serine residue. Interestingly, DegP is not inhibited by the classic serine protease inhibitor, phenylmethylsulfonylfluoride (PMSF), suggesting differences in the mode of action of DegP (Kolmar, “DegP or Protease DO CLAN SA” in Handbook of Proteolytic Enzymes, Barrett et al., eds., Academic Press, Great Britain (1998), Lipinska et al. (1990)). Site-directed mutagenesis at serine 210 and histidine 105, two components of the serine protease catalytic triad, compromised Deg function in vitro and in vivo; i.e., strains carrying serine 210 or histidine 105 mutant derivatives were sensitive for growth at elevated temperatures (Skorko-Glenek et al., Gene 163:47-52 (1995)). A recent study has shown that DegP functions as both a chaperone and a protease; at low temperatures (28° C.) the chaperone function predominates and after a shift to high temperature (45° C.) the protease function is activated (Spiess et al., Cell 97:339-347 (1999)). Synthesis of DegP is controlled by σ^{E}, the so-called “stress” sigma factor that controls genes essential for survival in the face of extracellular stress (Raina et al., J. Bacteriol. 175(8):5009-5021 (1993); Rouviere et al., EMBO J. 14:1032-1042 (1995)). This response is regulated, in part, by the CpxA/CpxR two-component regulatory system (Danese and Silhavy, Genes & Develop. 11:1183-1193 (1997); Danese et al., Genes & Develop. 9:387-398 (1995)). Recently, Jones et al. (Jones et al., EMBO J. 16:6394-6406 (1997)) demonstrated that expression of pilin subunits in the absence of the cognate periplasmic chaperone (such conditions result in pilin subunit aggregation) resulted in activation of the degP promoter.

The first identified in vivo target for DegP was colicin A lysis protein (Cal) (Cavard et al., J. Bacteriol. 171:6316-6322 (1989)). DegP was found to clip the acylated precursor form of Cal into two fragments. Mature Cal also accumulated to higher levels in degP mutant strains (Cavard et al., 1989). A second family of DegP targets identified were bacterial pilins. The K88 and K99 pilin subunits were found to accumulate to higher levels in degP mutant strains (Bakker et al., Mol. Microbiol. 5(4):875-886 (1991)). A more detailed study of this phenomenon demonstrated that P pilins, specifically PapA and PapG, are substrates for the DegP protease (Jones et al., 1997). The H. influenzae non-pilus adhesin proteins HMW1 and HMW2 were also found to be in vivo substrates for DegP (St. Geme III and Grass, Mol. Microbiol. 27:617-630 (1998)). In addition, Spiess et al. (Spiess et al., 1999) recently demonstrated that the MalS protein of E. coli was fully degraded in vitro by DegP.

In the last several years, a significant body of data has accumulated demonstrating that DegP is a virulence factor for several pathogenic organisms. In Salmonella typhinurium, Salmonella typhi, Brucella abortus, Brucella melitensis and Yersinia enterocolitica htrA nulls were found to reduce or abolish virulence (Elzer et al., Res. Veterin. Sci. 60:48-50 (1996a), Elzer et al., Infect. Immun. 64:4838-4841 (1996b); Johnson et al., Mol. Microbiol. 5:401-407 (1991); Li et al., Infect. Immun. 64:2088-2094 (1996); Phillips et al., Res. Veterin. Sci. 63:165-167 (1997); Tacket et al., Infect. Immun. 65:452-456 (1997)). The htrA null mutants were found to be more sensitive to oxidative stress and killing by immune cells. Moreover, an htrA lesion was found to be useful in attenuating both Salmonella typhi (Tacket et al., 1997; Tacket et al., Infect. Immun. 68(3):1196-1201 (2000)) and Salmonella typhimurium (Roberts et al., Infect. Immun. 68(10):6041-6043 (2000)) for implementation as vaccine strains. Boucher et al. (Boucher et al., J. Bacteriol. 178:511-523 (1996)) demonstrated that Pseudomonas aeruginosa conversion to mucoidy, the so-called cystic fibrosis phenotype, involved two htrA homologues. In a recent report, Pederson et al. (Pederson et al., Infect. Immun. 69(4):2569-2579 (2001)) demonstrated that and htrA null mutant in Legionella pneumophilia was attentuated for virulence. Recently, degP was insertionally inactivated in Streptococcus pyogenes and found to result in temperature and oxidative sensitivity as well as compromising virulence in a mouse model (Jones et al., 2001).

Early in vivo data suggested that pilin subunits of the so-called chaperone-usher assembly pathway were DegP substrates (Bakker et al., 1991). Expression of pilin subunit proteins in the absence of the cognate-chaperone (PapD) resulted in failure to accumulate subunits in the periplasm of wild-type (DegP+) bacteria and degP mutant strains were found to accumulate higher levels of pilin subunit in the periplasm (Bakker et al., 1991; Hultgren et al., “Bacterial Adhesins and Their Assembly” in Escherichia coli and Salmonella: Cellular and Molecular Biology, F. C. Niehardt, ed., ASM Press, Washington D.C., pp. 2730-2756 (1996); Hultgren et al., Annu. Rev. Microbiol. 45:383-415 (1991); Jones et al., 1997). Both the toxicity and accumulation was suppressed by complementation with papD, encoding the periplasmic chaperone, or degP (Jones et al., 1997). Previously, we developed a scheme for purification of the PapA subunit of the P pilus and demonstrated that this protein is a natural substrate that is efficiently cleaved by the DegP protease. In the current invention, we also identify three cleavage sites within the PapA sequence and characterized the determinants essential for proteolytic cleavage of one of the peptide substrates.

### SUMMARY OF THE INVENTION

The present invention provides for methods of treatment and/or prophylaxis of diseases caused by pilus-forming bacteria by modulating DegP protease activity. The present invention provides for the identification of DegP cleavage sites on PapA pilin subunit and methods for identifying substances which modulate DegP activity. The present invention provides for polypeptides identified which are cleavable substrates for DegP. The present invention provides for the identification of a polypeptide which enhances DegP protease activity.

### BRIEF DESCRIPTION OF THE DRAWINGS

FIG. 1 Expression and purification of PapA-6H4A.

1A. Accumulation of PapA-6H4A in the periplasm is dependent on the PapD chaperone. Periplasmic fractions prepared from KS474/pCL101/pHJ9203 were analyzed following IPTG (1 mM) induction of PapA-6H4A alone (lane 1) or co-induction of both PapA-6H4A and PapD (lane 2). Cultures were induced for 60 minutes at mid-logarithmic growth. Periplasm was fractionated on SDS-PAGE and stained with Coomassie blue.

1B. Purification of PapZ-6H4A-PapD complex from the periplasm. Periplasm from double induced cultures was applied to Talon metal affinity resin (Clontech). The pre-Talon, unbound fraction and first three washed (20 mM Tris, pH=8, 100 mM NaCl) are shown in lanes 2-6, respectively. Talon resin containing bound PapD-PapA-6H4A complex was treated with 8 M urea and washed to remove PapD (lanes 7-9). The bound PapA-6H4A was then eluted with 0.1 M imidazole, 8 M urea (lanes 10-12). In order to remove the trace of PapD contaminating the PapA-6H4A (lanes 10-12), the material in the three elution fractions was pooled, dialyzed, and reapplied to Talon resin in 20 mM Tris, pH=8, 8 M urea, following extensive washes, PapA-6H4A was eluted with 0.1 M imidazole, 8 M urea (lane 13). Lane 1 contains molecular weight markers.

1C. Reconstitution of PapD-PapA-6H4A complex. Denatured PapA-6H4A (lane 2) was applied to Talon metal affinity resin and diluted into a solution containing purified PapD chaperone. The resin was washed and the chaperone-subunit complex eluted with 0.1 M imidazole (lane 3). Lane 1 contains purified native PapD PapA-6H4A comples for reference.

1D. DegP protease binding and cleavage of PapA-6H4A. Denatured PapA-6H4A was lined to metal affinity resin and incubated for 60 minutes at room temperature with control periplasm (KS272/pACYC184, lane 1) or periplasm enriched with DegP protease (KS272/pKS17, lane 2). Following three washes, bound protein was eluted with 0.1 M imidazole. Three novel bands are apparent following exposure of PapA-6H4A to DegP-enriched periplasm. The ˜48 kDa band (lane 2) was identified by amino terminal sequence analysis as the DegP protease while the approximately 12 kDa band (lane 2) has the expected amino terminus for PapA-6H4A. The third band, which runs above the indicated 14 kDa molecular weight marker, has yet to be identified. Lane 3 contains molecular weight markers. In A-D, the sizes of molecular weight markers are indicated.

FIG. 2. Toxicity Assays.

2A. PapA toxicity suppression by PapD. Ks474/pCL101/pHJ9203 (papD) was induced with 0.3 mM IPTG to induce synthesis of PapA-6H4A at the onset of growth in the presence (▪) or absence (▴) of PapD. PapD was induced with 4% arabinose at the onset of culture growth. The A₆₀₀ of each culture was monitored throughout growth.

2B. PapA toxicity suppression by DegP. KS474/pCL101/pKS17 (degP) (▪) and KS474/pCL101/pACYC184 (vector control) (▴) were induced with 0.3 mM IPTG at the onset of growth. The A₆₀₀ of each culture was monitored throughout growth

FIG. 3. Purification of DegP.

3A. Cation exchange fractionation. Periplasm prepared from 30 grams of cells was applied to a 5 mL HiTrap SP column and eluted with a linear salt gradient. The starting material and flow-through fraction are shown in lanes 2 and 3, respectively. The relevant portion of the elution gradient is shown in lanes 4-8. DegP eluted at approximately 100 mM NaCl.

3B. HIC butyl fractionation. Peak fractions from the cation exchange fractionation were pooled, brought to 0.5 M ammonium sulfate and applied to a HiTrp HIC butyl column. The flow-through fraction is shown in lane 2 and a portion of the gradient elution is shown in lanes 3-8. DegP eluted in approximately 0.3 M NaCl and is shown in lanes 4-8. The small arrows indicated truncated forms of DegP, all of which were identified by amino-terminal sequencing (unpublished data). In both A and B, lane 1 contains high molecular weight markers.

FIG. 4. In vitro Protease Assay.

Pooled peak HIC butyl fractions from three separate DegP purification runs (A, B, C) were checked for general protease activity on a commercial casein substrate. The EnzChek assay kit uses a sensitive fluorescein labeled casein substrate that is internally quenched until cleaved. The BODIPY-casein FL signal is read following excitation at 485 nm and monitoring 530 nm emission. Peak fractions containing DegP were incubated with the substrate according to manufacturer's instructions and after one hour were read in a plate fluorometer. Trypsin was used as a control for the assay.

FIG. 5. In vitro DegP Cleavage Assay.

5A. Reduced and carboxymethylated PapA-6H4A (PapA-6H4A-rcm) was mixed with DegP in 20 mM Tris, pH=8, and incubated overnight at 45° C. The reactions were resolved on SDS-PAGE, transferred to PVDF membrane and developed with a polyclonal antibody raised against whole P pili. PapA-6H4A-rcm (lanes 2 & 3, 0.25 μg, lanes 4 & 5, 0.5 μg) was incubated in the presence (lanes 3 & 5) and absence (lanes 2 &4) of approximately 50 and 100 fold molar excess of DegP (lanes 3 & 5, respectively). The large arrowhead indicates a PapA aggregate in lane 4. Densitometric quantification of the cleaved PapA-6H4A band indicated that DegP cleaved 84% of the input protein (compare lanes 2 & 3) and nearly 100% of the aggregate band and 49% of the monomer in lane 5. Lane 1 contains DegP alone as a control. Some cross-reactivity between PapA antisera and DegP was seen in the blot.

5B. Addition of divalent cation, MgCl₂, enhances DegP cleavage of PapA. Reduced and carboxymethylated PapA-6H4A (20 μg) was mixed with DegP (20 μg) in 20 mM Tris, pH=8, 5 mM MgCl₂ and incubated for 4 hours at 45° C. The reactions, containing DegP alone (lane 1), PapA-6H4A-rcm alone (lane 3) and DegP+PapA-6H4A-rcm (lane 2) were resolved on SDS-PAGE and stained with Coomassie brilliant blue. Nearly 50% of the input PapA-6H4A-rcm was cleaved during the 4-hour incubation.

FIG. 6. DegP Cleavage of PapA-6H4A Substrate Is Inhibited by Chelating Agent and Stimulated by Addition of a Non-Cleavable Peptide, SPCJ-1.

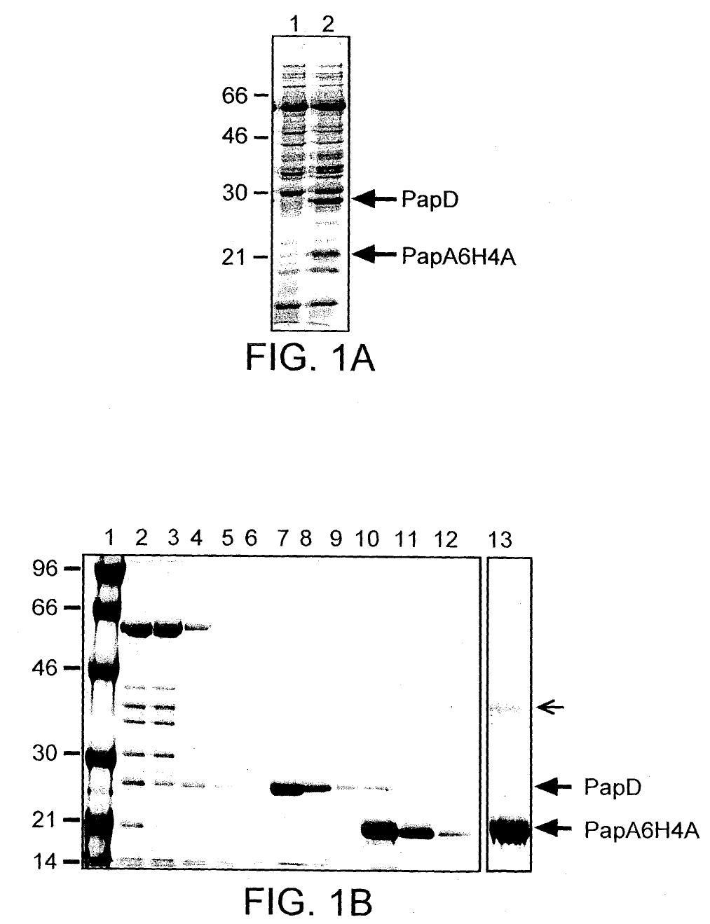

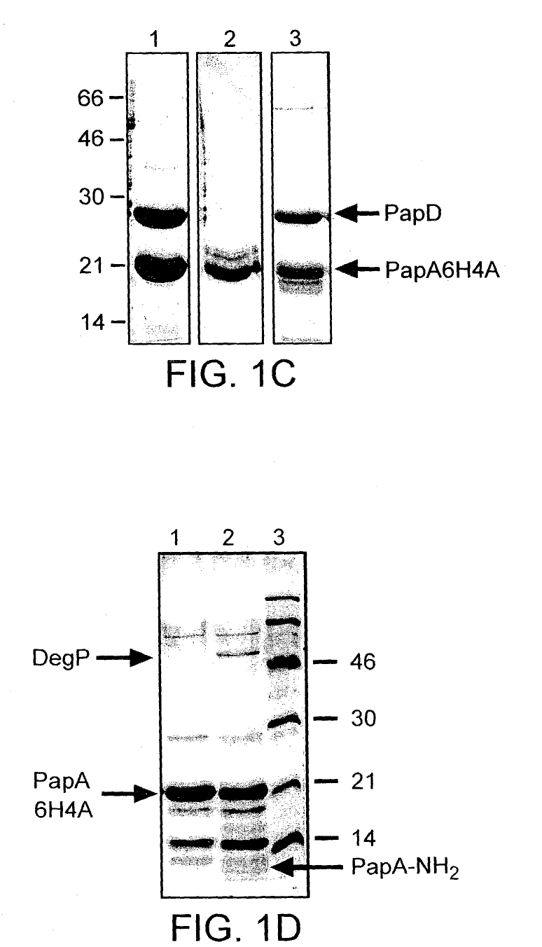

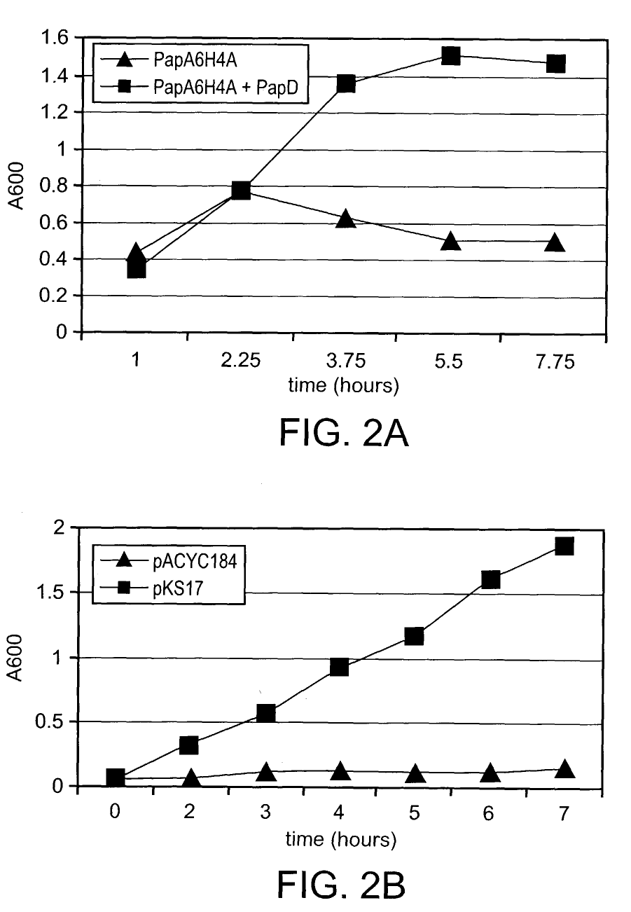

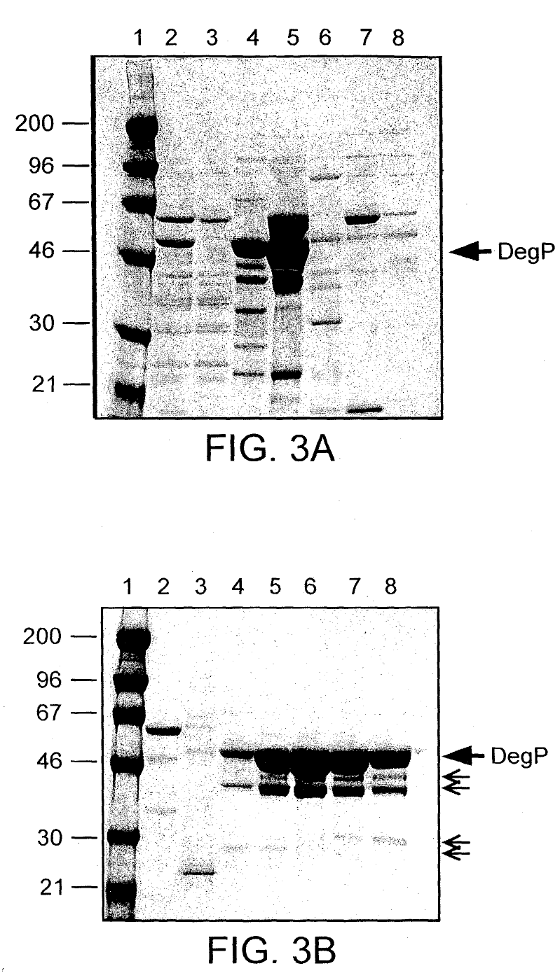

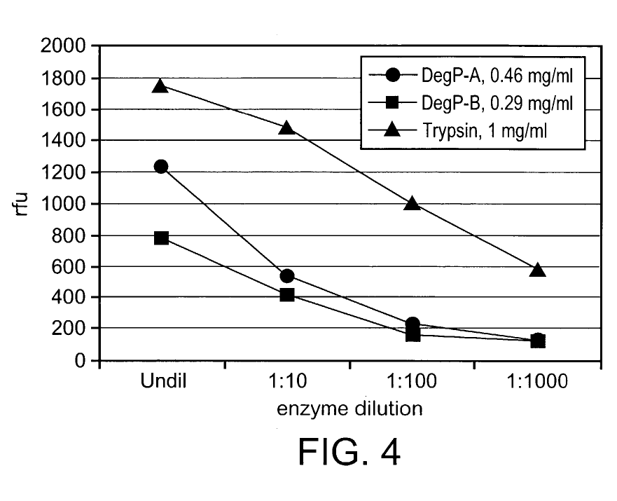

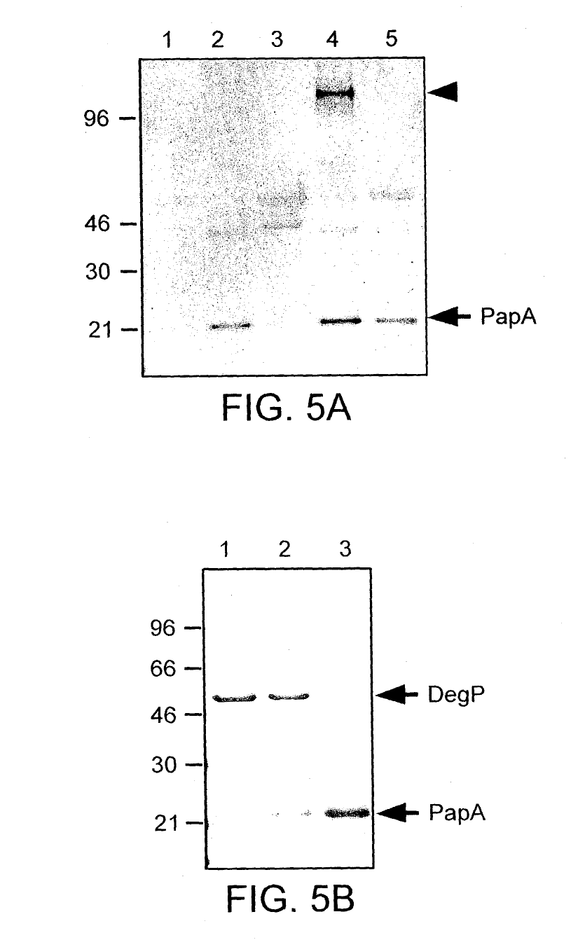

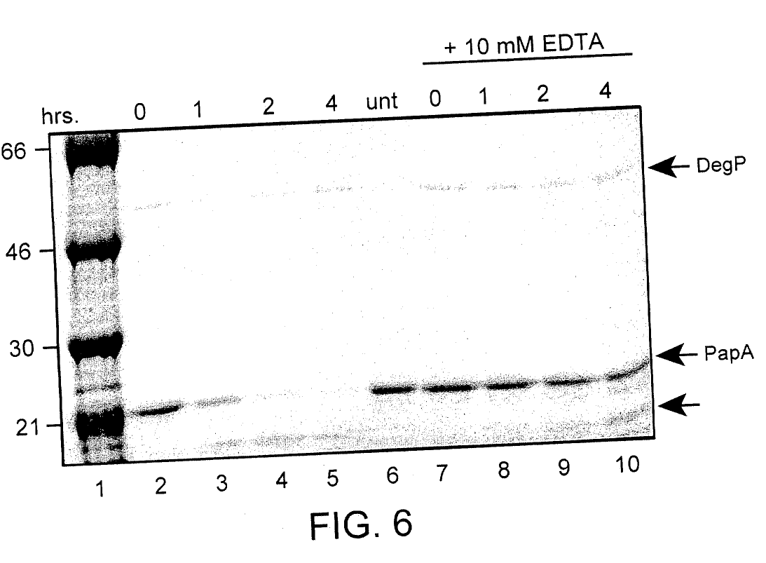

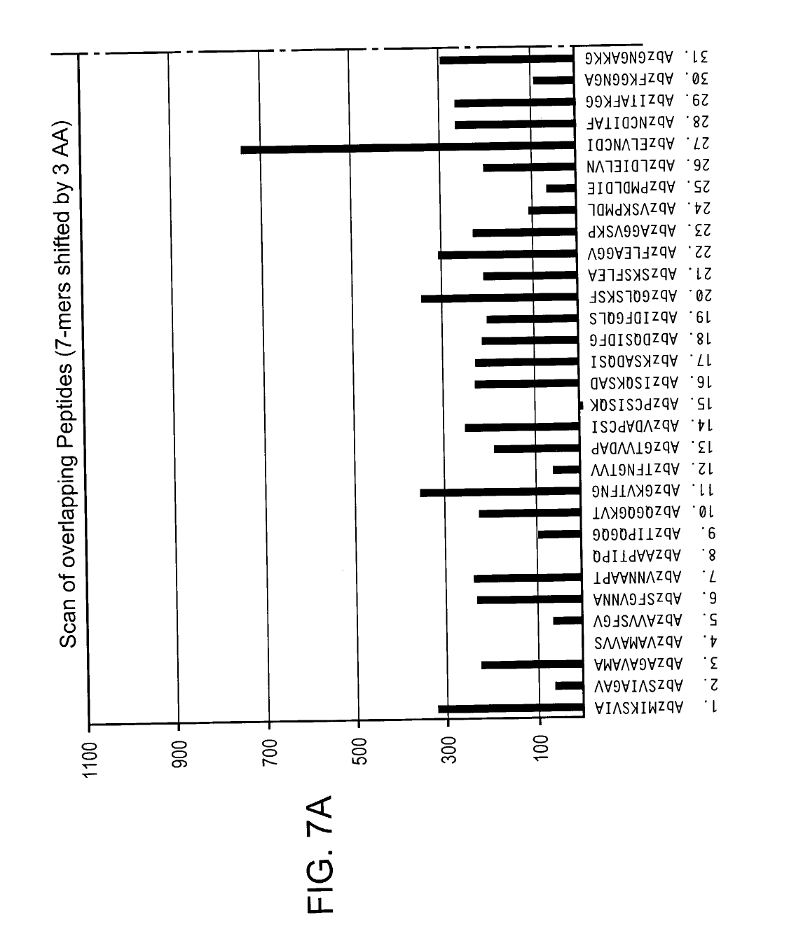

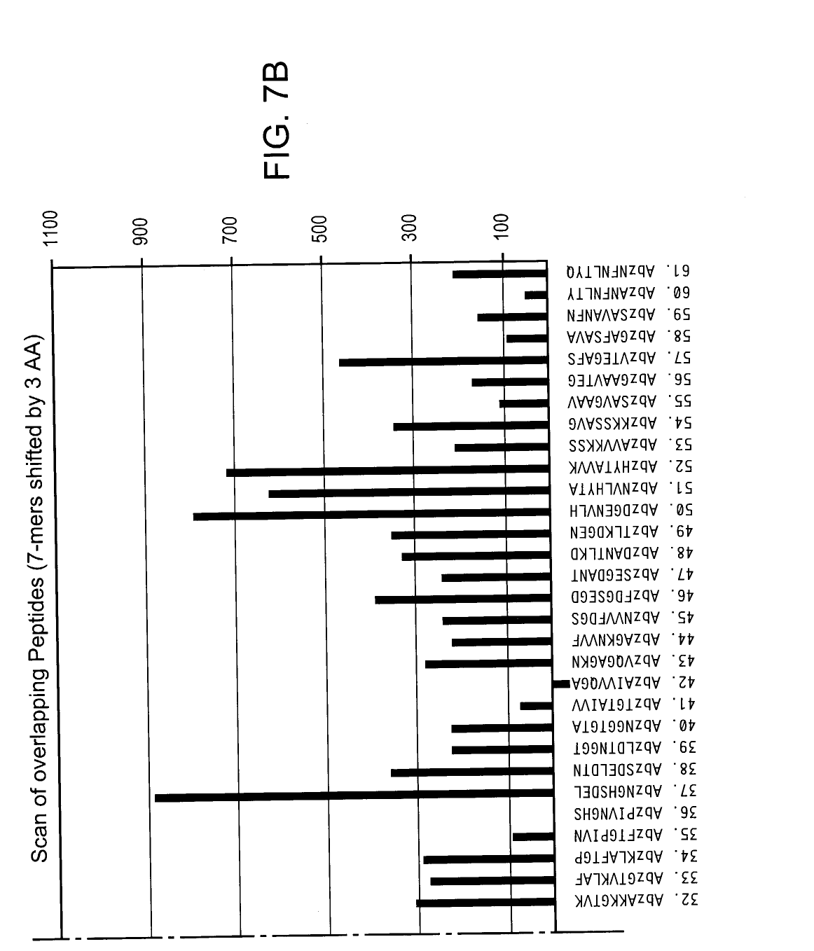

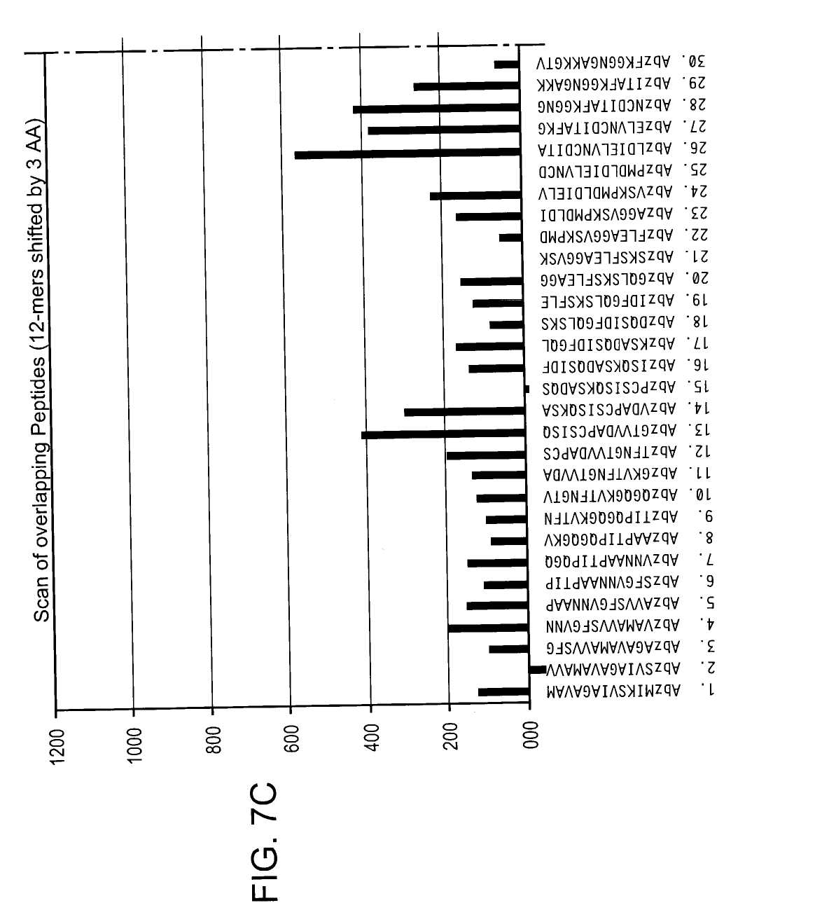

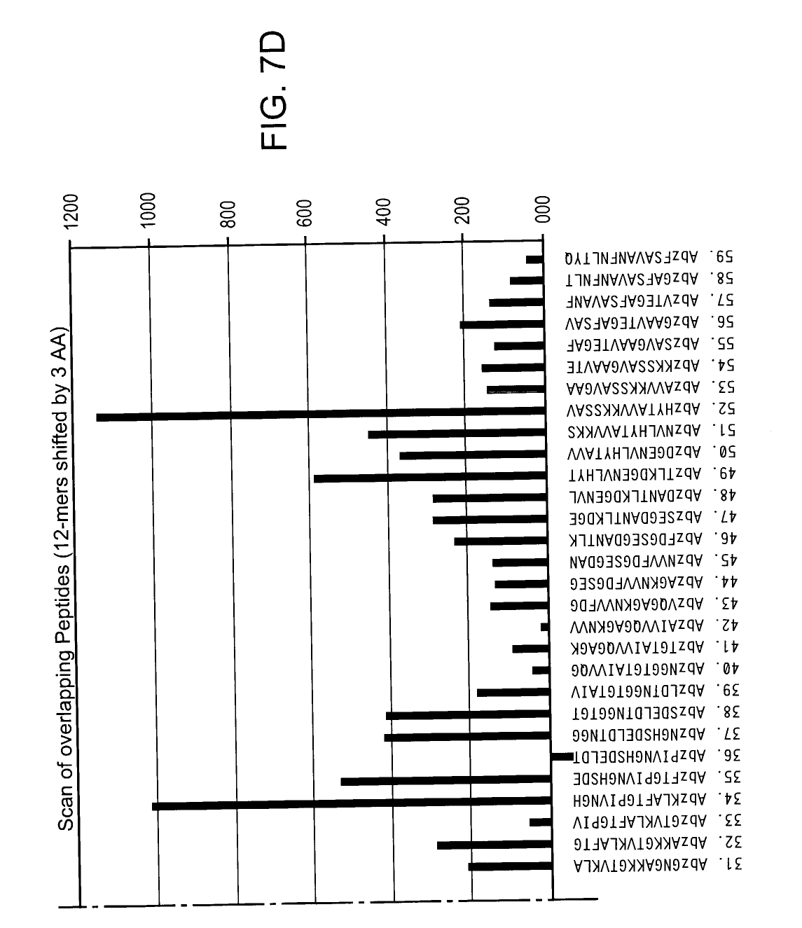

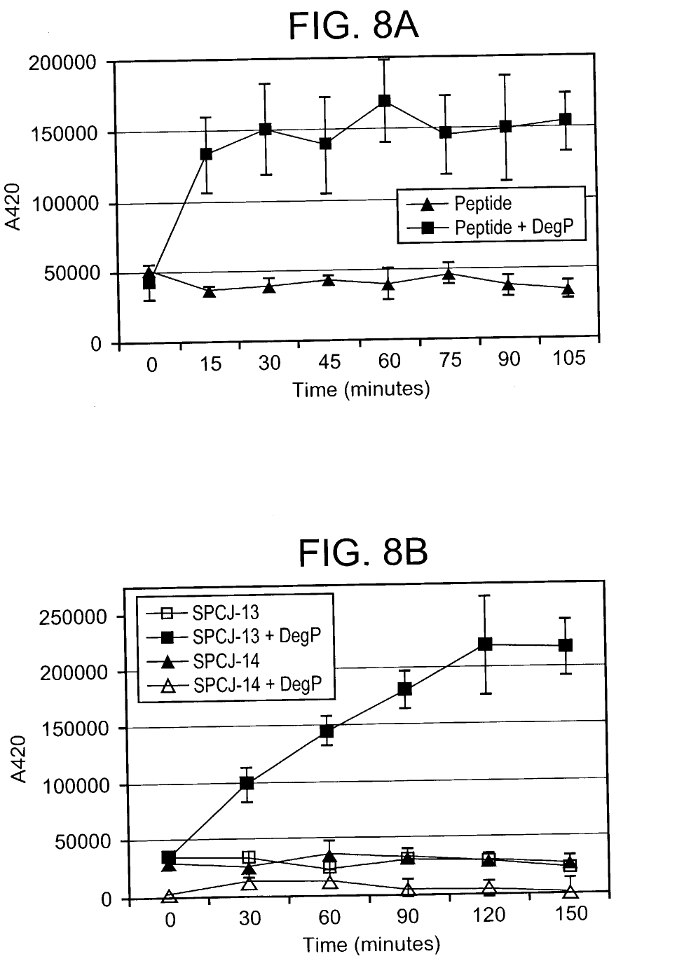

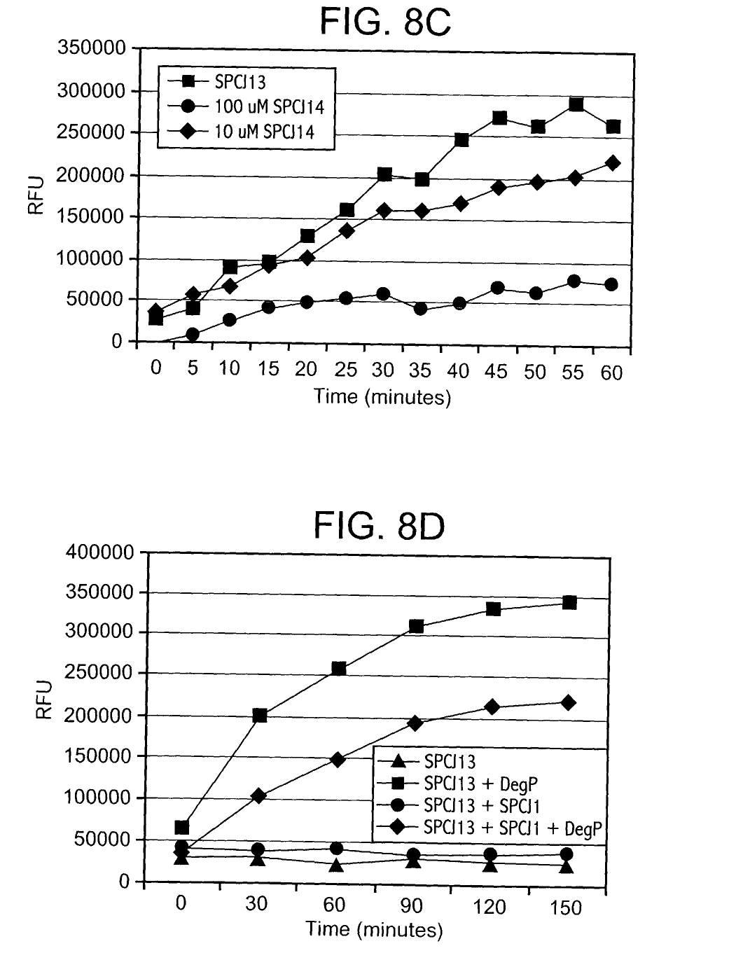

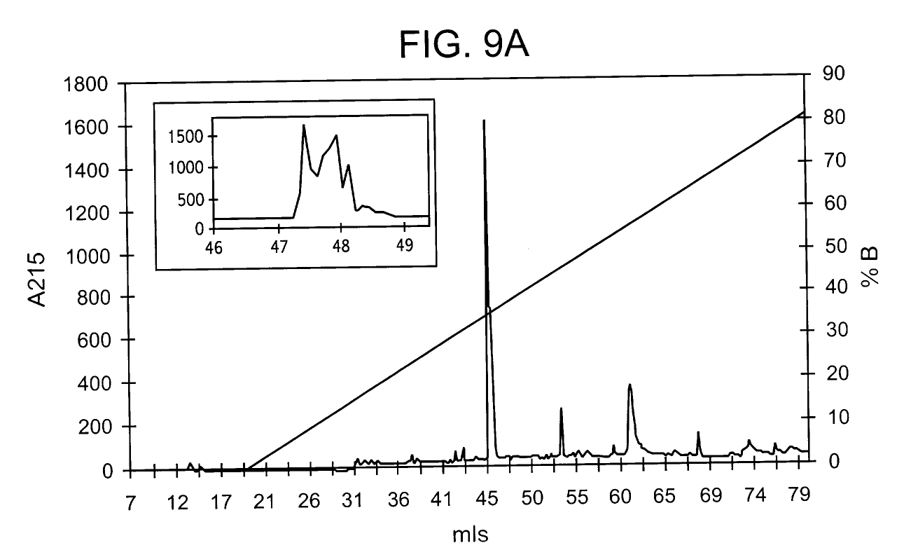

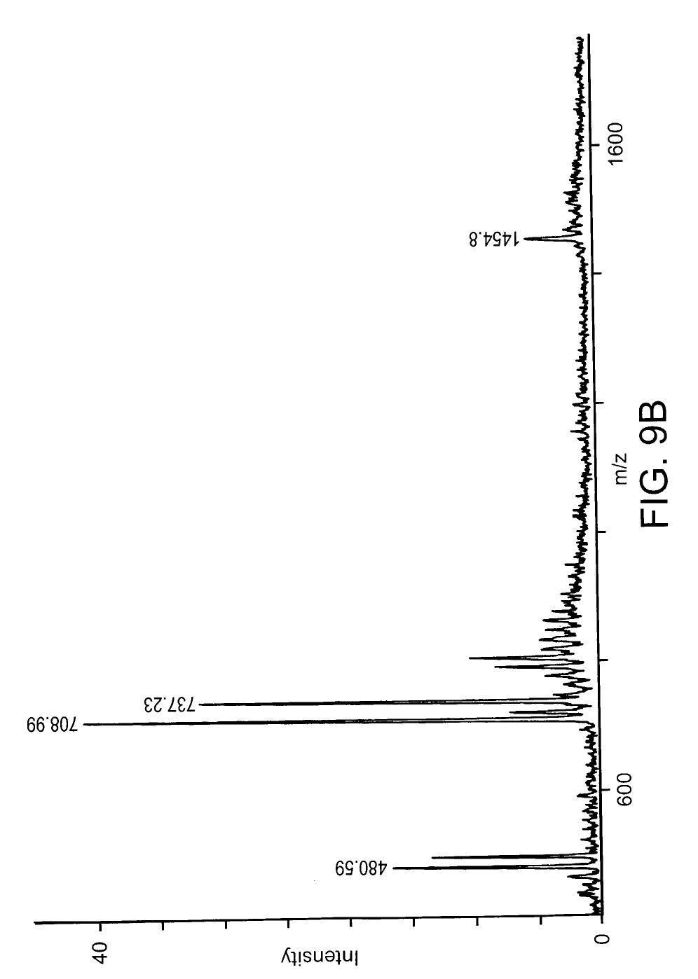

DegP (5 μg) was incubated with carboxymethylated PapA-6H4A (40 μg) in 20 mM Tris, pH=8, 5 mM MgCl₂ in the presence of 200 μM SPCJ-1. EDTA (10 mM) was added to the reactions loaded in lanes 7-10. Aliquots were taken at the indicated time points and the reaction stopped by the addition of SDS-loading buffer and incubation on ice. The reactions were resolved on SDS-PAGE and stained with Coomassie brilliant blue. The ˜12 kDa cleavage product (lanes 3-5) seen in earlier cleavage assays (FIG. 1D) is indicated by the arrowhead. In excess of 50% of input PapA-6H4A was degraded in 60 minutes and 90% at the four hour time point.

FIG. 7. Identification of the DegP Protease Cleavage Site in PapA.

SPOT synthesis technology was used to construct two overlapping (3 residues) peptide libraries, 7-mer and 12-mer, of the PapA sequence. The peptides were synthesized linked to a continuous cellulose membrane (ProteaseSpots) and had a fluorescent tag, aminobenzoic acid (Abz) at the amino-terminus. The PepSpots were assayed in a 96-well format with 0.28 mg/ml DegP in 20 mM PO₄, pH=7.5, 5 mM MgCl₂. The data presented are the liberated fluorescence (excitation—325 nm, emission—420 nm) following 28 hours incubation. The data show relative levels of cleavage for each of the peptides in the 7-mer (FIG. 7A) and 12-mer (FIG. 7B) libraries.

FIG. 8. In vitro Peptide Cleavage Assay.

8A. SPCJ-12 (8 μM) was incubated with, 5 μg (▪), and without (▴) DegP in reaction buffer (25 mM HEPES, pH=8,5 mM CaCl₂) for the indicated times.

8B. SPCJ-13 (▪, □) and SPCJ-14 (▴, Δ) (10 μM) were incubated with, 5 μg (▪, ▴), and without, (□, Δ), DegP in reaction buffer for the indicated times.

8C. Inhibitory activity of SPCJ-14 was demonstrated by pre-incubating DegP, 5 μg, with 10 μM (♦) or 100 μM (●) SPCJ-14 for 30 minutes and then adding 10 μM SPCJ-13 and continuing the incubation for 60 minutes.

8D. SPCJ-13 was incubated with, 5 μg (▪, ♦), and without, (●, ▴), DegP in reaction buffer. To test the stimulatory effect of SPCJ-1, 200 μM peptide was added (▪, ●) at the onset of incubation. Assays were monitored (excitation—340 nm, emission—420 nm) at the indicated times in a Victor² V plate reader.

FIG. 9. Mapping DegP Cleavage Site in Substrate Peptides.

9A. RP-HPLC was employed to resolve products of a scaled-up (5 ml) cleavage reaction. The peak (actually two peaks—see insert), at 45.72 ml, that showed absorbance at 215 nm, 340 nm, and 420 nm was lyophilized to dryness and the substituents identified by MALDI TOF Mass Spectroscopy.

9B. The single peak contained two species resulting from the cleavage of the peptide. The first species had a m/z of 480.59 corresponding to VK-DAP(Dnp)-NH₂ (SEQ ID NO.: 1) while the other species had a m/z of 708.99 corresponding to Abz-HYTAV (SEQ ID NO.: 2). This analysis was repeated using the SPCJ-12 peptide (12-mer) and mapped the same cleavage site (data not shown).

### DETAILED DESCRIPTION OF THE PREFERRED EMBODIMENTS

In general, terms in the present application are used consistent with the manner in which those terms are understood in the art.

By “modulate” is meant methods, conditions, or agents which enhance, increase, inhibit, or decrease the wild-type activity of an enzyme and the like.

By “modulated activity” is meant any activity, condition, disease or phenotype which is modulated by a protein. This modulation may take place by e.g., by direct agonistic or antagonistic effect as, for example, through inhibition, activation, binding, or release of substrate, modification either chemically or structurally, or by direct or indirect interaction which may involve additional factors. This change may be an increase/decrease in catalytic activity and/or binding to substrates.

By “DegP” is meant a multifunctional protein essential for the removal of misfolded and aggregated proteins in the periplasm of E. coli. DegP also refers to HtrA and Protease Do in the current invention. “DegP homologue” refers to other proteases, such as DegQ and DegS, which are other E. coli. proteins with high homology to DegP. Although the Examples refer to DegP of E. coli, the current invention is not limited to this organism and is intended to encompass other Gram-negative homologues of DegP, chlamydia, and certain Gram-positive bacteria possessing DegP homologues.

The current invention provides for a method for identifying a substance that modulates the protease activity of DegP or a DegP homologue by adding said substance to DegP or a DegP homologue in the presence of a cleavable substrate, and detecting enhancement or inhibition of the cleavage of said cleavable substrate, thereby determining whether said substance modulates said protease activity.

In a preferred embodiment, the cleavable substance used in this method is a polypeptide selected from the group consisting of HYTAVVKKSSAV (SEQ ID NO: 3), HYTAVVK (SEQ ID NO: 4), LDIELVNCDITA (SEQ ID NO: 5), ELVNCDI (SEQ ID NO: 6), KLAFTGPIVNGH (SEQ ID NO: 7), FTGPIVNGHSDE (SEQ ID NO: 8), TLKDGENVLHYT (SEQ ID NO: 9), DGENVLH (SEQ ID NO: 10), NVLHYTA (SEQ ID NO: 11), and NGHSDEL (SEQ ID NO: 12), which are PapA pilin subunit peptides identified as cleaved by DegP.

In a preferred embodiment the DegP homologue is DegS or DegQ.

In another preferred embodiment, the substance of this method enhances protease activity. In another preferred embodiment, the substance inhibits protease activity. In a particularly preferred embodiment, the substance modulates DegP activity.

The current invention provides for a method for treatment or prophylaxis of disease caused by pilus-forming bacteria, comprising preventing, inhibiting, or enhancing the protease activity of DegP or a DegP homologue. It will be apparent to one skilled in the art from the foregoing Background section and the Example below, that DegP is a virulence factor which is responsible for the degradation of denatured or aggregated proteins from the periplasmic space. Therefore, inhibition of DegP protease activity may lead to toxic buildup of pilin subunits in the periplasmic space, which may lead to increased morbidity and/or mortality or decreased infectiveness of a bacterium. However, enhancement of DegP protease activity may also have deleterious consequences for a bacterium, such as depletion of necessary pilin subunits. Thus, either enhancement or inhibition of DegP protease activity may reduce the pathogenicity of pilus-forming bacteria.

In the current invention, cleavage sites on the PapA pilin subunit have been identified which are efficiently cleaved by DegP protease. Accordingly, the current invention provides for isolated polypeptides comprising the amino acid sequences HYTAVVKKSSAV (SEQ ID NO: 3), HYTAVVK (SEQ ID NO: 4), LDIELVNCDITA (SEQ ID NO: 5), ELVNCDI (SEQ ID NO: 6), KLAFTGPIVNGH (SEQ ID NO: 7), FTGPIVNGHSDE (SEQ ID NO: 8), TLKDGENVLHYT (SEQ ID NO: 9), DGENVLH (SEQ ID NO: 10), NVLHYTA (SEQ ID NO: 11), and NGHSDEL (SEQ ID NO: 12). These sequences were identified in assays performed in the current invention as DegP protease sites on the PapA pilin subunit. It will be appreciated by one skilled in the art that the peptides of the current invention may be modified, e.g., with aminobenzoic acid and/or diaminopropionamide dinitrophenyl, and still perform according to the current invention. Any modified peptides may be tested, e.g., in the assays detailed in the Example, to determine whether such modification affects the activity of the peptide in modulating DegP protease activity. It was observed, as is noted in the Example below, that in most cases, cleavable substrates of DegP identified contained paired hydrophobic residues.

The amino acid compounds of the invention are polypeptides which are partially defined in terms of amino acid residues of designated classes. Polypeptide homologs would include conservative amino acid substitutions within the amino acid classes described below. Amino acid residues can be generally sub-classified into four major subclasses as follows:

Acidic: The residue has a negative charge due to loss of H⁺ ion at physiological pH, and the residue is attracted by aqueous solution so as to seek the surface positions in the conformation of a peptide in which it is contained when the peptide is in aqueous medium, at physiological pH.

Basic: The residue has a positive charge due to association with H⁺ ion at physiological pH, and the residue is attracted by aqueous solution so as to seek the surface positions in the conformation of a peptide in which it is contained when the peptide is in aqueous medium at physiological pH.

Neutral/non-polar: The residues are not charged at physiological pH, but the residue is repelled by aqueous solution so as to seek the inner position in the conformation of a peptide in which it is contained when the peptide is in aqueous medium. These residues are also designated “hydrophobic.”

Neutral/polar: The residues are not charged at physiological pH, but the residue is attracted by aqueous solution so as to seek the outer positions in the conformation of a peptide in which it is contained when the peptide is in aqueous medium.

It is understood, of course, that in a statistical collection of individual residue molecules some molecules will be charged, and some not, and there will be an attraction for or repulsion from an aqueous medium to a greater or lesser extent. To fit the definition of “charged”, a significant percentage (at least approximately 25%) of the individual molecules are charged at physiological pH. The degree of attraction or repulsion required for classification as polar or nonpolar is arbitrary and, therefore, amino acids specifically contemplated by the invention have been classified as one or the other. Most amino acids not specifically named can be classified on the basis of known behavior.

Amino acid residues can be further subclassified as cyclic or noncyclic, and aromatic or non-aromatic, self-explanatory classifications with respect to the side chain substituent groups of the residues, and as small or large. The residue is considered small if it contains a total of 4 carbon atoms or less, inclusive of the carboxyl carbon. Small residues are, of course, always nonaromatic.

The secondary amino acid proline, although technically within the group neutral/nonpolar/large/cyclic and nonaromatic, is a special case due to its known effects on the secondary conformation of peptide chains, and is not, therefore, included in this defined group.

Other amino acid substitutions can also be included in peptide compounds within the scope of the invention and can be classified within this general scheme according to their structure.

All of the compounds of the invention may be in the form of the pharmaceutically acceptable salts or esters. Salts may be, for example, Na⁺, K⁺, Ca⁺², Mg⁺² and the like; the esters are generally those of alcohols of 1-6 carbons.

The current invention provides for a method for modulating the protease activity of DegP or a DegP homologue comprising adding one or more substances selected from the group consisting of a non-cleavable substrate or a cleavable substrate to DegP or a DegP homologue in the presence of a cleavable substrate in an amount sufficient to modulate said protease activity.

In a preferred embodiment, the DegP homologue is DegQ or DegS. In another preferred embodiment, protease activity is inhibited. In another embodiment, the modulating substance is a cleavable substrate. In a particularly preferred embodiment, the cleavable substrate is a polypeptide selected from the group consisting of HYTAVVKKSSAV (SEQ ID NO: 3), HYTAVVK (SEQ ID NO: 4), LDIELVNCDITA (SEQ ID NO: 5), ELVNCDI (SEQ ID NO: 6), KLAFTGPIVNGH (SEQ ID NO: 7), FTGPIVNGHSDE (SEQ ID NO: 8), TLKDGENVLHYT (SEQ ID NO: 9), DGENVLH (SEQ ID NO: 10), NVLHYTA (SEQ ID NO: 11), and NGHSDEL (SEQ ID NO: 12). In another preferred embodiment, protease activity is enhanced. In a particularly preferred embodiment, said substance is a polypeptide having amino acid sequence KSMCMKLSFS (SEQ ID NO: 13).

The present invention provides for a composition of matter, comprising a substance which modulates the protease activity of DegP or a DegP homologue, and a carrier therefor. This composition of matter may have diagnostic or pharmaceutical use. For example, small molecule inhibitors of DegP or DegP homologue function identified by an assay of the current invention can be used as novel anti-infectives. Further, small peptide molecules such as those identified in the present invention as cleavage sites can be used as fusion protein “linkers.” Such linkers stabilize proteins which are difficult to produce for overexpression and purification in E. coli. DegP or a DegP homologue can then be used to cleave the fusion protein during purification.

In a preferred embodiment, the substance of the composition of matter comprises a polypeptide comprising an amino acid sequence selected from the group consisting of HYTAVVKKSSAV (SEQ ID NO: 3), HYTAVVK (SEQ ID NO: 4), LDIELVNCDITA (SEQ ID NO: 5), ELVNCDI (SEQ ID NO: 6), KLAFTGPIVNGH (SEQ ID NO: 7), FTGPIVNGHSDE (SEQ ID NO: 8), TLKDGENVLHYT (SEQ ID NO: 9), DGENVLH (SEQ ID NO: 10), NVLHYTA (SEQ ID NO: 11), and NGHSDEL (SEQ ID NO: 12). In another preferred embodiment, the composition of matter comprises a polypeptide comprising amino acid sequence KSMCMKLSFS (SEQ ID NO: 13). In another embodiment, the composition of matter further comprises at least one antibacterial agent, wherein said agent is selected from the group consisting of penicillins, cephalosporins, aminoglycosides, sulfonamides, tetracyclines, chloramphenicol, polymixins, antimycobacterial drugs, and urinary antiseptics.

Substances that are assayed by the above disclosed methods can be randomly selected or rationally selected or designed. As used herein, substance is said to be randomly selected when the agent is chosen randomly without considering the specific sequences involved in the association of DegP alone or with its associated substrates, etc. An example of randomly selected agents is the use of a chemical library or a peptide combinatorial library, or a growth broth of an organism.

The substances of the present invention can be, as examples, peptides, small molecules, vitamin derivatives, as well as carbohydrates. A skilled artisan can readily recognize that there is no limit as to the structural nature of the agents of the present invention.

The peptide substances of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production of polypeptides using solid phase peptide synthesis is necessitated if non-nucleic acid-encoded amino acids are to be included.

### EXAMPLE

The following example is intended to demonstrate preferred embodiments of the invention and is not considered to be limiting.

### Identification of DegP Cleavage Sites and Proteolysis of PapA and Enhancement of DegP Protease Activity

Materials and Methods

Strains and Genetic Constructs

DH5α (Hanahan, J. Mol. Biol. 166:557-580 (1983)) and INFαF′ (Invitrogen, Carlsbad, Calif.) were used in cloning steps. KS272 (MC1000 [F-Δ(ara-leu)7697 galE galK ΔlacX74 rpsL (str^{r})] was used for expression of DegP (Strauch et al., J. Bacteriol. 171:2689-2696 (1989)). KS474 (KS272 degP::kan) was used for production of PapA-6H4A. pKS17 (Strauch et al., (1989)), a gift of T. Silhavy, was used for overexpression and purification of DegP. PCR cloning of papA was performed as previously described (Morrison and Desrosiers, BioTechniques 14(3):454-457 (1993)) using primers designed to insert six histidine codons immediately downstream of the PapA leader processing site. The mutated papA gene was amplified to include BamHI and EcoRI restriction sites to facilitate cloning into pMMB566 (Furste et al., Gene 48(1):119-131 (1986)) creating pCL100. pCL101 was constructed in the same fashion using primers designed to insert six histidine codons and two alanine codons downstream of the leader processing site of the papA gene. The PapD expression plasmid, pHJ9203, was described previously (Jones et al., EMBO J. 16:6394-6406 (1997)).

PapA-6H4A Expression and Purification

KS474/pCL101/pHJ9203 was grown in Luria-Bertani (LB) broth (Difco, Becton-Dickinson, Sparks, Md.) in the presence of 100 μg/ml ampicillin, 50 μg/ml kanamycin, 100 μg/ml spectinomycin until A₆₀₀=0.8, at which time IPTG was added to 1.0 mM and arabinose was added to 0.5%. The culture was induced for 90 minutes, the bacteria harvested and periplasm prepared from approximately 25 grams (wet weight) of cells as previously described (Jones et al., 1997). The periplasm was dialyzed against 20 mM Tris, pH=8.5, and applied to Talon metal affinity beads in batch as described by the manufacturer (Clontech, Palo Alto, Calif.). Following incubation for 60 minutes at RT and three washed with 20 mM Tris, pH=8, 100 mM NaCl the bound complex was eluted with 0.1 M imidazole, or alternatively, the chaperone-subunit complex was denatured by resuspension in 20 mM Tris, pH=8.0, 100 mM NaCl, 6.0 M guanidine-HCl (or 8.0 M urea) and incubation at 45° for 60 minutes. Guanidine-HCl was found to work best in our hands for disruption of the chaperone-subunit complex. The chaperone was removed by washing in buffer and denaturant and PapA-6H4A eluted from the Talon resin with 0.1 M imidazole. For efficient cleavage by DegP the PapA-6H4A was subjected to reduction and carboxymethylation. The guanidine-HCl-denatured PapA-6H4A was reduced in a buffer consisting of 400 mM Tris, pH=8.5, 5.0 mM EDTA, 0.11 M 2-mercaptoethanol and incubated at 4° C. for two hours. The reduced protein was carboxymethylated by incubation with 0.21 M iodoacetic acid for one hour at 4° C. The protein was then dialyzed into 20 mM Tris, pH=8.5, for use in cleavage assays.

Purification of DegP Protease

DegP was purified from whole periplasm, which was prepared as previously described (Jones et al., 1997). Approximately 30 grams (wet weight) of cells were used to prepare periplasm. Following dialysis against 33 mM MES, 33 mM HEPES, 33 mM acetate, pH=5.9, the periplasm was applied to a HiTrap S cation exchange column (Amersham-Pharmacia Biotech, Uppsala, Sweden). DegP eluted at approximately 100 mM NaCl in a linear salt gradient. Peak protein fractions were adjusted to 0.5 M ammonium sulfate and applied to an HIC butyl column (Amersham-Pharmacia). DegP eluted from the hydrophobic column at approximately 0.3 M ammonium sulfate. All six bands appearing in the peak fractions eluting from the butyl column were identified as DegP species by amino-terminal sequence anaylsis.

Whole Protein Cleavage Assay

Proteolysis assays were performed in a buffer consisting of 20 mM Tris, pH=8, at 45° C. for 30 minutes to 15 hours. 5 mM MgCl₂ or CaCl₂ was added to later assays and to verify dependence on divalent cations, EDTA was added at 10 mM. Where indicated, the assay was supplemented (200 μM) with the non-cleavable peptide, SPCJ1, having the sequence KSMCMKLSFS, which is derived from the carboxy-terminus of the PapG adhesin.

Peptide Scanning (PepSpots™)

Overlapping peptide scanning libraries of PapA sequence in a 7-mer and 12-mer format and shifted by 3 amino acid residues were prepared by the SPOT-synthesis technique, conjugated to aminobenzoic acid at the amino-terminus, linked (carboxyl-terminus) to continuous cellulose membranes (ProteaseSpots™) and assayed by Jerini AG (Berlin, Germany). Purified DegP was prepared as described above and assayed (0.28 mg/ml) in a 96-well microplate format using 20 mM phosphate buffer, pH=7, 5 mM MgCl₂. At 1, 4, and 28 hours, aliquots were removed from the assay plate and liberated fluorescence quantified (excitation—325 nm, emission—420 nm).

Peptide Cleavage Assay

The following three peptides were used in the fluorescence-proximity peptide cleavage assay:

(SEQ ID NO:14)

SPCJ-12 - Abz-HYTAVVKKSSAV-DAP(Dnp)-NH₂

(SEQ ID NO:15)

SPCJ-13 - Abz-HYTAVVK-DAP(Dnp)-NH₂

(SEQ ID NO:16)

SPCJ-14 - Abz-HYTASSK-DAP(Dnp)-NH₂

where Abz, the fluorescent group, is aminobenzoic acid and DAP(Dnp)-NH₂, the quench group, is diaminopropionamide dinitrophenyl. The peptides were used in the assay at concentrations ranging from 200 pM to 1 nM. DegP was added into the assay at concentrations ranging from 0.5 to 5 μg. The assay buffer consisted of 25 mM HEPES, pH=7.5, 5 mM CaCl₂. The cleavage reactions were incubated at 45° C. and read in a Wallac Victor²V 1420 Multilabel HTS Counter (Wallac Oy, Turku, Finland) at 30-minute intervals. Fluorescence detection utilized excitation at 340 nm and emission was monitored at 420 nm.

Cleavage Site Mapping

Purification of peptide and products of cleavage reaction was performed on a C18 5μST 4.6/250 Sephasil column (Amersham-Pharmacia Biotech) with a gradient to 100% acetonitrile/0.05% trifluoroacetic acid. Lyophilized cleavage reactions (5 ml) were resuspended in 2% acetonitrile/0.065% trifluoroacetic acid for application to the C18 column. Fractions containing peptide and cleavage products were lyophilized to dryness and mass determined by MALDI TOF Mass Spectroscopy. MALDI analysis was conducted using 5 mg/ml solution of the matrix ?-cyano-4-hydroxycinnamic acid (HCCA) in 0.1% trifluoroacetic acid, 33% acetonitrile. Analyte was mixed with the matrix at a ratio of 1:3. Spectra were produced using a custom-built MALDI-TOF mass spectrometer.

Other Methods

Proteins were prepared for sequencing by transfer to PVDF membrane as previously described (Dodson et al., Proc. Natl. Acad. Sci. USA 90:3670-3674 (1993); Slonim et al., EMBO J. 11(13):4747-4756 (1992)) and delivered to Midwest Analytical Inc. (St. Louis, Mo.) for amino terminal sequence determination. SDS-PAGE and Western blot analysis were performed as previously described (Dodson et al., 1992; Slonim et al., 1992). Toxicity assays were performed as previously described (Jones et al., 1997). The EnzChek Assay Kit (Molecular Probes, Eugene, Oreg.) was used according to manufacturer's instructions and read using a Tecan SpectrFluor Plus (Research Triangle Park, N.C.) with the appropriate filter sets.

Results

Construction and Purification of PapA-6H4A

A significant obstacle to the study of chaperone-mediated pilus biogenesis is the inability to purify pilin subunits in the absence of the periplasmic chaperone. This can be overcome, in part, by inactivation of the DegP protease (Bakker et al., Mol. Microbiol. 5(4):875-886 (1991)). However, pilin subunits from the P (Pap) pilus were found to be highly toxic when expressed, absent the chaperone, in KS474 (degP::kan)(Jones et al., 1997). By taking advantage of metal-affinity chromatography under denaturing conditions and the addition of an affinity tag to the amino-terminus of PapA, the pilin subunit was produced and purified. Using the method of Morrison & Desrosiers (1993), a sequence encoding a polyhistidine affinity tag (6-his tag) was inserted into the papA gene. The 6-his tag was positioned immediately downstream of the leader-processing site so that the leader-processed protein would contain an exposed amino-terminal 6-his tag for affinity purification. The fusion protein was cloned into pMMB66 placing it under control of the IPTG inducible P_{tac} promoter (Furste et al., 1986). The initial construct, PapA-6H, was not appropriately processed or transported to the periplasm (data not shown). We postulated that the 6-his affinity tag was sterically blocking either leader processing or association with the Sec membrane transport machinery. This block was circumvented by the addition of two alanine residues, resulting in a total of four alanine residues, positioned to separate the 6-his tag from the leader-processing site in PapA. PapA-6H4A expressed from this construct, pCL101, was processed appropriately and transported to the periplasm (FIG. 1). Moreover, PapA-6H4A was dependent on an interaction with the periplasmic chaperone, PapD, for stability in the periplasm (FIG. 1A). As with the wildtype PapA, the PapA-6H4A protein was toxic when induced in the absence of the PapD chaperone (Jones et al., 1997). The toxicity is manifested in the failure of bacteria to grow following induction of the pilin subunit (FIG. 2). Earlier studies showed that “unchaperoned” subunits resided in the inner-membrane fraction and formed insoluble aggregates (Jones et al., 1997). The toxicity of PapA-6H4A was suppressed by co-expression of papD (FIG. 2A) or degP (FIG. 2B) in trans.

In order to purify sufficient PapA for the planned experiments, PapA-6H4A was synthesized in the presence of the PapD chaperone in KS474 (degP::kan)(Jones et al., 1997). The PapD-PapA complex was purified from whole periplasm using metal affinity chromatography (MAC) in batch (FIG. 1B). In order to separate the chaperone and the PapA-6H4A subunit the bead-bound complex was denatured with 6.0 M guanidine HCl. Following washes with 10 mM Tris, pH=8, plus denaturant to remove the chaperone, pure PapA-6H4A was eluted with 0.1 M imidazole (FIG. 1B). To verify that the denatured PapA-6H4A was a suitable binding partner for PapD, the chaperone-subunit complex was reconstituted and purified on MAC (FIG. 1C). Denatured PapA-6H4A was applied to the metal affinity resin and washed, although the denaturant was not removed. The bead-bound protein was then diluted into either PapD-enriched periplasm (data not shown) or purified PapD (FIG. 1C). The chaperone-subunit complex was washed and eluted from the affinity resin with 0.1 M imidazole (FIG. 1C, lane 3).

In order to test the denatured PapA-6H4A protein as a substrate for DegP protease, the resin-bound, denatured PapA-6H4A was diluted into DegP-enriched periplasm or control periplasm and incubated for 60 minutes at room temperature. Bound proteins were eluted with 0.1 M imidazole and analyzed by Coomassie Blue staining and amino-terminal sequencing of the eluted products (FIG. 1D). Two novel bands were identified. The ˜48 kDa band was identified as the DegP protease. An ˜12 kDa band (PapA-NH₂), that appears only following treatment with DegP-enriched periplasm, was identified as an amino-terminal fragment of PapA. This data defines an interaction between PapA and DegP that resulted in cleavage of PapA.

Purification of DegP Protease

DegP was purified from KS272/pKS17 (Strauch et al., 1989) following overnight (15-20 hours) growth to saturation, which was sufficient to induce high-level expression of DegP. Whole periplasm prepared from six liters of culture was fractionated by cation exchange chromatography on SP sepharose (HiTrap SP, Amersham-Pharmacia Biotech)(FIG. 3A) followed by hydrophobic interaction chromatography on butyl sepharose (HiTrap butyl, Amersham-Pharmacia Biotech)(FIG. 3B). This two-step purification process resulted in approximately 98% pure DegP protease. The “contaminating bands” (small arrows) seen on SDS-PAGE analysis shown in FIG. 3 were identified as DegP truncates by amino-terminal sequence analysis and presumably result from autocatalytic cleavage (data not shown). Incubation of the purified preparations of DegP at 45° C. resulted in accumulation of the truncate bands (data not shown).

Assay of Protease Activity

A commercially-available protease assay, EnzChek (Molecular Probes, Eugene, Oreg.) was used to test cleavage activity of the DegP preparations on a casein substrate. DegP activity on casein was previously described by Lipinska et al. (J. Bacteriol. 172:1791-1797 (1990)). Peak cleavage on the EnzChek substrate followed the elution profile of DegP through both chromatography steps (data not shown). FIG. 4 shows a titration of purified DegP protease and trypsin on the casein substrate.

Soluble PapA Cleavage Assay

Previous studies of DegP cleavage activity indicate that the preferred substrates were denatured, aggregated, or unfolded proteins (Kim et al., J. Mol. Biol. 295(5):1363-1374 (1999); Kolmar et al., J. Bacteriol. 178:5925-5929 (1996); Pallen and Wren, Mol. Microbiol. 26(2):209-221 (1997)). To provide DegP with a suitable substrate for cleavage in a soluble format, PapA-6H4A was denatured with 6 M guanidine-HCl, reduced and carboxymethylated (PapA-6H4A-rcm) and dialyzed into 20 mM Tris, pH=8.5. PapA-6H4A-rcm was mixed with DegP and incubated at 45° C. overnight and the resulting reaction products resolved on SDS-PAGE and further visualized by Western blot using antisera prepared against whole Pap pili (FIG. 5). As seen in FIG. 5A lane 4, PapA-6H4A-rcm forms polymers (or aggregates) that are stable in SDS, in addition to the 21 kDa monomer. Both forms of PapA-6H4A-rcm are sensitive to degradation by DegP protease (FIG. 5A, lanes 3 and 5).

Previous reports have described several different buffer systems suitable for monitoring DegP proteolytic activity (Kim et al., 1999; Kolmar et al., 1996; Lipinska et al., 1990; Spiess et al., Cell 97:339-347 (1999)). Divalent cation (Mg²⁺) was reported to be required in some systems (Kim et al., 1999). Therefore, we tested the effect of MgCl₂, MnCl₂, and CaCl₂ on DegP cleavage of PapA-6H4A-rcm (FIG. 5B and data not shown). Addition of 5 mM MgCl₂ (FIG. 5B), MnCl₂, or CaCl₂ (data not shown) stimulated DegP cleavage of PapA-6H4A-rcm resulting in nearly 50% of the input protein being degraded in a four-hour incubation (FIG. 5B). Moreover, addition of EDTA to the cleavage reaction inhibits cleavage activity (FIG. 6, compare lanes 3-5 and 7-10).

Activation of DegP by a Carboxyl-Terminal Pilin Subunit Peptide

The DegP protease has two so-called PDZ domains (Post-synaptic density 95, Discs-large, ZO-1) located downstream of the catalytic serine residue (Pallen & Wren, 1997). PDZ domains have been implicated in both substrate recognition and protein multimerization in a number of proteins (Levchenko et al., Cell 91:939-947 (1997); Sassoon et al., Mol. Microbiol. 33:583-589 (1999); Songyang et al., Science 275:73-77 (1997)). Levchenko et al. (1997) recently demonstrated that the ClpX chaperone and regulatory subunit of the ClpXP protease machine utilizes PDZ domains in substrate recognition. The PDZ domains specifically recognize disordered peptide sequences found at the carboxyl-terminus of target proteins (Levchenko et al., 1997; Songyang et al., 1997). The carboxyl-terminus of pilin subunits is a highly conserved motif important for interaction with neighboring subunits in the ultimate structure of the pilus as well as a recognition motif for the periplasmic chaperone (Bullitt et al., Proc. Natl. Acad. Sci. USA 93:12890-12895 (1996); Choudhury et al., Science 285(5430):1061-1066 (1999); Hultgren et al., Bacterial Adhesins and Their Assembly in Escherichia coli and Salmonella: Cellular and Molecular Biology, pages 2730-2756, F. C. Niehardt, ed., ASM Press, Washington, D.C. (1996); Sauer et al., Science 285(5430):1058-1061 (1999)). The carboxyl-terminal motif has some homology to the motif reportedly recognized by PDZ domains (Levchenko et al., 1997; Songyang et al., 1997). We hypothesized that titatraion of the carboxyl-terminal peptide from the PapG adherence protein (SPCJ-1-KSMCMKLSFS) (SEQ ID NO: 13) into the DegP cleavage reaction would inhibit cleavage of the PapA-6H4A-rcm protein through competition for the substrate binding site in the PDZ domain. Much to our surprise, addition of SPCJ-1 to the cleavage reaction enhanced degradation of the PapA-6H4A-rcm (FIG. 6). In the absence of SPCJ-1 50% cleavage of the PapA protein required a four-hour digestion at 45° C. (FIG. 5B and data not shown), whereas addition of the activating peptide resulted in 50% cleavage in 60 minutes and nearly 90% cleavage at four hours in a reaction containing four-fold less enzyme and twice as much substrate (FIG. 6, lanes 2-5). As expected, cleavage of PapA-6H4A-rcm was inhibited (7% cleavage in 1 hour) in the presence of 10 mM EDTA (FIG. 6, lanes 7-10).

Identification of DegP Recognition Sites Within PapA by Peptide Scanning

Utilizing SPOT synthesis technology (Jerini A G, Berlin, Germany), two overlapping peptide scanning libraries of the complete PapA amino acid sequence were constructed and assayed to identify 7-mer and 12-mer peptides that were suitable substrates for DegP proteolysis (FIG. 7). The peptides carried an aminobenzoic acid (Abz) fluorescent tag at the amino terminus and were synthesized linked to a cellulose membrane. In both the 7-mer and the 12-mer ProteaseSpot™ scans, 3 regions of PapA were identified as having a sequence cleavable by DegP as represented by release of fluorescent counts from the cellulose bound peptides. Interestingly, the cleavable regions identified by this analysis all lie within or proximal to β-strands and are in highly conserved regions of pilin subunit proteins (see FIG. 4 in Soto and Hultgren, J. Bacteriol. 181:1059-1071 (1999)).

Peptide Cleavage Assay

In order to establish a soluble peptide cleavage assay to monitor DegP protease activity, the most efficiently cleaved region, represented by the sequence—HYTAVVKKSSAV (SEQ ID NO: 3)—was used as a model substrate and a peptide, SPCJ-12, prepared for use in a fluorescent-quench detection assay. SPCJ-12 was prepared with an aminobenzoic acid (Abz) group, the fluorescent moiety, on the amino-terminus and a diaminopropionamide dinitrophenyl (DAP(Dnp)-NH₂) group, the quench moiety, on the carboxyl terminus. As shown in FIG. 8A, DegP cleaved SPCJ-12 in a time-dependent fashion. To further narrow the recognition sequence and define the determinants of cleavage, two additional peptide reagents, SPCJ-13 and SPCJ-14, were prepared. SPCJ-13 has the sequence HYTAVVK (SEQ ID NO: 4) (the first 7 residues of SPCJ-12), whereas SPCJ-14 has the sequence HYTASSK (SEQ ID NO: 17). The double serine replacement was utilized to test the essentiality of the paired hydrophobic residues in SPCJ-13. As shown in FIG. 8B, DegP cleaved SPCJ-13 in a time-dependent fashion, however, SPCJ-14 was not cleaved. In order to gain insight into the failure of DegP to cleave the double serine replacement peptide, we set up an inhibition assay to see if SPCJ-14 could block cleavage of SPCJ-13. Such a result would suggest that DegP still recognized SPCJ-14 but could not cleave the peptide. As shown in FIG. 8C, when added in 10-fold molar excess, SPCJ-14 blocked cleavage of SPCJ-13. Lastly, addition of the non-cleavable peptide SPCJ-1, to the peptide cleavage assay enhanced cleavage of SPCJ-13 (FIG. 8D).

In order to map the precise cleavage sites in SPCJ-12 and SPCJ-13, the reaction products of the cleavage reactions were resolved on Reverse-Phase HPLC using a C18 Sephasil column (FIG. 9A). Untreated SPCJ-13 had a retention volume of 53.06 mls (data not shown), whereas after cleavage a single new peak was seen with a retention volume of 45.72 mls and the 53.06 mls peak was greatly diminished (FIG. 9A). The mass of the material in the new peak was determined by MALDI-TOF Mass Spectroscopy analysis. The new peak contained two species having masses (480.59=VK-DAP(Dnp)-NH₂; 708.99=ABZ-HYTAV) consistent with cleavage between the paired valine residues in the peptide (FIG. 9B). As a confirmation, the cleavage site in SPCJ-12 was also mapped by MALDI MS and again it was demonstrated that DegP cleaved between the paired valine residues (data not shown).

Discussion

The degradation and clearance of misfolded and/or misassembled proteins in the cytoplasm and periplasm is essential for vigorous growth of bacteria (Gottesman, Ann. Rev. Genet. 30:465-506 (1996); Laskowska et al., Mol. Microbiol. 22:555-571 (1996); Pallen & Wren, 1997). In the periplasm of E. coli and most Gram-negative organisms this task falls to the DegP protease (Pallen & Wren, 1997). Absent a functional copy of degP, bacteria fail to grow at high temperature (Lipinska et al., 1990; Pallen & Wren, 1997). Moreover, recent data shows that in addition to compromising growth at high temperature, degP null mutants are more sensitive to oxidative stress and are avirulent (Boucher et al., J. Bacteriol. 178:511-523 (1996); Elzer et al., Res. Vet. Sci. 60:48-50 (1996a); Elzer et al., Infect. & Immun. 64:4838-4841 (1996b); Li et al., Infect. & Immun. 64:2088-2094 (1996); Pallen & Wren, 1997; Pederson et al., Infect. & Immun. 69(4):2569-2579 (2001);Phillips et al., Res. Vet. Sci. 63:165-167 (1997); Roberts et al., Infect. & Immun. 68(10):6041-6043 (2000); Tacket et al., Infect. & Immun. 65:452-456 (1997); Tacket et al., Infect. & Immun. 68(3):1196-1201(2000)). The current model suggests that the oxidative response of the host to an invading pathogen results in oxidative denaturation of proteins in the periplasm. The inability of a degP mutant strain to clear or refold denatured protein places a sufficient burden on the pathogen resulting in a reduction in virulence in several recently described animal models of infection (Boucher et al., 1996; Elzer et al., 1996a; Elzer et al., 1996b; Li et al., 1996; Pallen & Wren, 1997; Pederson et al., 2001 ;Phillips et al., 1997; Roberts et al., 2000; Tacket et al., 1997; Tacket et al., 2000).

In keeping with this model, the preferred substrate for DegP appears to be proteins that are globally or transiently denatured, supporting the hypothesis that the role in vivo is to remove misfolded or denatured proteins from the periplasm (Kolmar et al., 1996). Kolmar et al. (Kolmar et al., 1996) demonstrated that DegP would cleave slow folding mutants of λ repressor and Arc repressor and that the cleavage site P1 residue was a hyrdophobic residue, most often a valine. These data support the hypothesis, since the hydrophobic cleavage site would only be available in denatured targets. Therefore, the recognition site for DegP substrates would be exposed when the proteins went “off pathway” or were denatured due to an environmental insult. Although an attractive model, this study was hampered by the fact that neither protein used is a periplasmic protein. In support of this finding, however, Laskowska et al. (Laskowska et al., 1996) demonstrated in vitro that purified DegP protein would degrade thermally aggregated proteins fractionated from E. coli extracts. Moreover, they showed that the DnaJ chaperone would antagonize DegP degradation; i.e., the chaperone would aid in refolding the proteins such that they were no longer targets for degradation by DegP.

We have clearly demonstrated that the PapA pilin, from the pyelonephritis-associated pilis (Pap) is an in vitro target for DegP. Using an amino-terminal poly-histidine affinity tag coupled with denaturing metal affinity chromatography, sufficient PapA-6H4A could be purified away from the periplasmic chaperone, PapD, for implementation in a soluble cleavage assay (FIG. 1). DegP protease was purified by standard chromatographic means (FIG. 3) and showed proteolytic activity on both a casein substrate as well as denatured, reduced, and carboxymethylated PapA-6H4A (FIGS. 4 and 5). DegP cleavage activity on PapA-6H4A that had not been reduced and carboxymethylated was minimal by comparison to its activity on PapA-6H4A-rcm (data not shown). However, defining the precise “folded state” of PapA absent complex formation with PapD or assembly into the pilus is a difficult proposition. Recent co-crystallization studies have revealed that pilin subunits have an incomplete Immunoglobulin fold due to the absence of a 7^{th} carboxyl-terminal strand needed to complete a β-sheet in the native fold (Choudhury et al., 1999; Sauer et al., 1999). During pilus biogenesis, this missing strand is provided by either the periplasmic chaperone or the neighboring pilin subunit through donor strand complementation or donor strand exchange, respectively (Choudhury et al., 1999; Sauer et al., 1999).

An amino-terminal truncate of the cleaved PapA was identified using the denatured substrate on the affinity resin as well as in the soluble cleavage assay, suggesting that cleavage occurred near the middle of the protein (FIGS. 1D and 6). To further define the specificity of DegP cleavage and to map the cleavage sites within PapA an overlapping peptide scan was utilized (ProteaseSpot™, Jerini A G, Berlin, Germany)(Reineke et al., High Throughput Screening Assay for the Identification of Protease Substrates in Peptides 2000: Proceedings of the 26^{th} European Peptide Symposium, J. Martinez & J. A. Fehrentz, eds. (2000)). Both scanning libraries (7-mer and 12-mer, FIG. 7) identified 3 regions of PapA that had enhanced cleavage by DegP in the solid-phase assay. The scanning results were verified for the carboxyl-terminal most peptide sequence, SPCJ-12 (HYTAVVKKSSAV) (SEQ ID NO: 3) and the shorter version, SPCJ-13 (HYTAVVK) (SEQ ID NO: 4) in a soluble cleavage assay utilizing a fluorescence-quench assay system (FIG. 8). The specificity of cleavage of this sequence was further defined using the control peptide, SPCJ-14 (HYTASSK) (SEQ ID NO: 17), which challenged the protease to cleave the peptide absent the paired valine residues. The lack of cleavage of SPCJ-14 is in agreement with previously reported studies from the Sauer laboratory that showed that the P1 residue of model DegP substrates was most often a valine residue (Kolmar et al., 1996). Of the other sequences identified by the peptide scanning, LDIELVNCDITA (SEQ ID NO: 5), ELVNCDI (SEQ ID NO: 6), KLAFTGPIVNGH (SEQ. ID NO: 7), FTGPIVNGHSDE (SEQ ID NO: 8), TLKDGENVLHYT (SEQ ID NO: 9), DGENVLH (SEQ ID NO: 10), and NVLHYTA (SEQ ID NO: 11) also have paired hydrophobic residues, however, the NGHSDEL sequence does not follow this pattern. Clearly, peptide structure, in addition to sequence, has a role to play in recognition by DegP as can be seem in several instances in which “neighboring” peptides which shared sequence were not equivalently cleaved in the solid-phase assay (FIG. 7).

The PapD cleavage fragment (˜12 kDa) identified in the initial solid-phase cleavage assay (FIG. 1D) was identified as an amino-terminal fragment of PapA by amino-terminal sequencing. This fragment again resulted in the solution phase assay (FIG. 6) and the identity of the cleavage product determined by amino-terminal sequencing. This cleavage product most likely results from cleavage at one of the upstream sites (LDIELVNCDITA) (SEQ ID NO: 5), (FTGPIVNGHSDE) (SEQ ID NO: 8).

In terms of substrate recognition, Sonyang et al., (Songyang et al., 1997) recently defined a carboxyl-terminal motif in target proteins recognized by PDZ domains. Considering this finding, PDZ domains in bacterial proteases may recognize a sequence that signals a misfolded state of the protein that differs from the protease cleavage site. The most striking feature of the motif identified by Songyang et al. is a conserved residue (threonine, serine, or tyrosine) at the −3 position (>60% of sequences identified) and a highly conserved (>80%) hydrophobic carboxyl terminal residue (valine, isoleucine, or leucine). Interestingly, an alignment of the carboxyl terminus of 44 pilin subunits revealed that the −3 position was often a serine, threonine, or tyrosine (˜50%)(Soto and Hultgren, 1999). Bacterial pili, such as Pap pili, assembled by the chaperone-usher assembly pathway were recently divided into two familes based on structural and sequence features: the so-called FGS and FGL subfamilies (Hung et al., 1996). In the FGL subfamily, the conservation at the −3 position (serine, threonine, or tyrosine) is greater than 80%. Our initial results with SPCJ-1 (KSMCMKLSFS) (SEQ ID NO: 13), which conforms to the carboxyl-terminal 10 residues of PapG, indicate that the PDZ domains in DegP play a role in substrate recognition through an interaction with the carboxyl-terminal sequence. Addition of SPCJ-1 and not an irrelevant peptide (data not shown) enhanced the cleavage activity of the protease (FIGS. 5, 6, & 8D). As described in detail in two recent papers (Choudhury et al., 1999; Sauer et al., 1999) and supported by earlier genetic data (Bullitt et al., 1996; Hultgren et al., 1996; Kuehn et al., 1993; Slonim et al., 1992; Soto and Hultgren, 1999; Xu et al., 1995) the carboxyl-terminus of pilin subunits is an integral component of the recognition site for chaperone-subunit complex formation. As discussed briefly above, the lack of the carboxyl-terminal 7^{th} strand of the immunoglobulin fold results in a deep groove on the surface of the pilin that exposes the hydrophobic core. One edge of this groove is lined by the carboxyl-terminal β-strand of the pilin (the F strand) (see (Choudhury et al., 1999; Sauer et al., 1999) for a complete discussion). During donor strand complementation, the chaperone donates its G strand to complete the immunoglobulin fold of the pilin. The donated G strand completes the hydrophobic core of the pilin through interactions with both sides of the groove. Both genetic data and molecular modeling support the model that the carboxyl-terminus of pilin subunits is part of the pilin-pilin interface that drives pilus assembly (Bullitt et al., 1996; Hultgren et al., 1996; Choudhury et al., 1999; Sauer et al., 1999). Therefore, this motif would rarely be exposed to the solvent. The exposure of this motif, in the event of misfolding or misassembly, would signal the need for either a chaperone to catalyze refolding or a protease to degrade the protein.

These studies illustrate the mechanism by which the major periplasmic protease, DegP, cleaves misfolded pilin subunits and may relate to the general proteolytic pathway for DegP substrates. DegP's role in the pathogenesis of both Gram-negative and Gram-positive pathogens provides the impetus to develop assays suitable for high-throughput screening and the identification of small molecule inhibitors of this important virulence target. The described studies pave the way for the development of a novel class of anti-infectives developed against the DegP protease.

All cited patents and publications referred to in this specification are herein incorporated by reference in their entirety.

We claim:

1. A method for identifying a substance that modulates the protease activity of DegP or a DegP homologue, said method comprising,
adding said substance to DegP or a DegP homologue in the presence of a cleavable substrate, and
detecting enhancement or inhibition of the cleavage of said cleavable substrate, thereby determining whether said substance modulates said protease activity.

2. The method of claim 1, wherein said cleavable substrate is a polypeptide selected from the group consisting of HYTAVVKKSSAV (SEQ ID NO: 3), HYTAVVK (SEQ ID NO: 4), LDIELVNCDITA (SEQ ID NO: 5), ELVNCDI (SEQ ID NO: 6), KLAFTGPIVNGH (SEQ ID NO: 7), FTGPIVNGHSDE (SEQ ID NO: 8), TLKDGENVLHYT (SEQ ID NO: 9), DGENVLH (SEQ ID NO: 10), NVLHYTA (SEQ ID NO: 11), and NGHSDEL (SEQ ID NO: 12).

3. The method of claim 1, wherein said DegP homologue is DegS or DegQ.

4. The method of claim 1, wherein said substance enhances said protease activity.

5. The method of claim 1, wherein said substance inhibits said protease activity.

6. The method of claim 1, wherein said substance modulates DegP activity

7. A method for treatment or prophylaxis of disease caused by pilus-forming bacteria, comprising preventing, inhibiting, or enhancing the protease activity of DegP or a DegP homologue.

8. An isolated polypeptide comprising an amino acid sequence selected from the group consisting of HYTAVVKKSSAV (SEQ ID NO: 3), HYTAVVK (SEQ ID NO: 4), LDIELVNCDITA (SEQ ID NO: 5), ELVNCDI (SEQ ID NO: 6), KLAFTGPIVNGH (SEQ ID NO: 7), FTGPIVNGHSDE (SEQ ID NO: 8), TLKDGENVLHYT (SEQ ID NO: 9), DGENVLH (SEQ ID NO: 10), NVLHYTA (SEQ ID NO: 11), and NGHSDEL (SEQ ID NO: 12).

9. A method for modulating protease activity of DegP or a DegP homologue, comprising,
adding one or more substances selected from the group consisting of a non-cleavable substrate or a cleavable substrate
to DegP or a DegP homologue in the presence of a cleavable substrate in an amount sufficient
to modulate said protease activity.

10. The method according to claim 9, wherein said DegP homologue is DegQ or DegS.

11. The method according to claim 9, wherein said protease activity is inhibited.

12. The method according to claim 9, wherein said substance is a cleavable substrate.

13. The method according to claim 12, wherein said cleavable substrate is a polypeptide selected from the group consisting of HYTAVVKKSSAV (SEQ ID NO: 3), HYTAVVK (SEQ ID NO: 4), LDIELVNCDITA (SEQ ID NO: 5), ELVNCDI (SEQ ID NO: 6), KLAFTGPIVNGH (SEQ ID NO: 7), FTGPIVNGHSDE (SEQ ID NO: 8), TLKDGENVLHYT (SEQ ID NO: 9), DGENVLH (SEQ ID NO: 10), NVLHYTA (SEQ ID NO: 11), and NGHSDEL (SEQ ID NO: 12).

14. The method according to claim 9, wherein said protease activity is enhanced.

15. The method according to claim 9, wherein said substance is a polypeptide comprising amino acid sequence KSMCMKLSFS (SEQ ID NO: 13).

16. A composition of matter, comprising a substance which modulates the protease activity of DegP or a DegP homologue, and a carrier therefor.

17. A composition of matter according to claim 16, wherein said substance is a polypeptide comprising an amino acid sequence selected from the group consisting of HYTAVVKKSSAV (SEQ ID NO: 3), HYTAVVK (SEQ ID NO: 4), LDIELVNCDITA (SEQ ID NO: 5), ELVNCDI (SEQ ID NO: 6), KLAFTGPIVNGH (SEQ ID NO: 7), FTGPIVNGHSDE (SEQ ID NO: 8), TLKDGENVLHYT (SEQ ID NO: 9), DGENVLH (SEQ ID NO: 10), NVLHYTA (SEQ ID NO: 11), and NGHSDEL (SEQ ID NO: 12).

18. A composition of matter according to claim 16, wherein said substance is a polypeptide having amino acid sequence KSMCMKLSFS (SEQ ID NO: 13).

19. A composition of matter according to claim 16, which further comprises at least one antibacterial agent, wherein said agent is selected from the group consisting of penicillins, cephalosporins, aminoglycosides, sulfonamides, tetracyclines, chloramphenicol, polymixins, antimycobacterial drugs, and urinary antiseptics.
